(19) United States
(12) Patent Application Publication
(10) Pub. No.: US 2009/0047177 A1
(43) Pub. Date: Feb. 19, 2009

(54) LACTATE CONCENTRATION MEASUREMENT DEVICE

(76) Inventors: Stephen D. Walker, Boulder, CO (US); Devon J. Dallmann, Boulder, CO (US); Charles W. Henry, Boulder, CO (US); Peter E. Nelson, Boulder, CO (US); R. Dale Zellers, Boulder, CO (US); John E. Repine, Boulder, CO (US)

Correspondence Address: KOESTNER BERTANI LLP, 2192 Martin St., Suite 150, Irvine, CA, 92612, US

(21) Appl. No.: 11/840,528

(22) Filed: Aug. 17, 2007

(51) Int. Cl. G01N 21/00; G01N 33/48

(52) U.S. Cl. 422/55; 436/63

(57) ABSTRACT

A lactate concentration measurement apparatus comprising a housing enclosing a sample chamber configured for holding a body fluid sample and measurement photo-optics that generate light and monitor light intensity along a plurality of optical paths in the sample chamber. The apparatus further comprises a plurality of optical filters aligned in respective optical paths of the optical path plurality comprising at least a first optical filter with light absorption by lactate and water and a second optical filter with light absorption to water alone. A logic determines lactate concentration based on a ratio of intensities detected at a first detector in an optical path intersected by the first optical filter and detected at a second detector in an optical path intersected by the second optical filter.

### BACKGROUND

Lactate is the metabolic product of glycolysis, formed from pyruvate in the cellular cytosol. Basal lactate production is 1 mmol/kg per hour for a 70 kg subject. Hyperlactatemia, elevated lactate levels, is common in disorders such as shock, low cardiac output, acute liver failure, severe sepsis, decompensated diabetes mellitus, cancer, AIDS, seizure, poisoning, drug therapy, and others. Lactate metabolism is difficult to assess since poor tissue perfusion from shock or sepsis produces more lactate whereas liver failure causes underutilization of lactate.

Exogenous lactate is added to the patient's blood and lactate measurements which are performed to determine the disorder, causing hyperlactatemia. Current hospital practice for measuring lactate involves a blood sample sent to central laboratory for analysis, a process that can take several hours, during which period the patient's health status can change dramatically.

### SUMMARY

An embodiment of a lactate concentration measurement apparatus comprises a housing enclosing a sample chamber configured for holding a body fluid sample and measurement photo-optics that generate light and monitor light intensity along a plurality of optical paths in the sample chamber. The apparatus further comprises a plurality of optical filters aligned in respective optical paths of the optical path plurality comprising at least a first optical filter with light absorption by lactate and water and a second optical filter with light absorption to water alone. A logic determines lactate concentration based on a ratio of intensities detected at a first detector in an optical path intersected by the first optical filter and detected at a second detector in an optical path intersected by the second optical filter.

### BRIEF DESCRIPTION OF THE DRAWINGS

Embodiments of the invention relating to both structure and method of operation may best be understood by referring to the following description and accompanying drawings:

FIG. 1 is a schematic block and pictorial diagram depicting an embodiment of a lactate concentration measurement apparatus;

FIGS. 2A through 2B are flow charts illustrating one or more embodiments or aspects of a method for measuring concentration of lactate in body fluid;

FIG. 3 is a schematic block and pictorial diagram showing another embodiment of a lactate concentration measurement apparatus;

FIGS. 4A through 4B are flow charts illustrating one or more embodiments or aspects of another method for measuring concentration of lactate in body fluid; and

FIG. 5 is a schematic block diagram showing an embodiment of a system that can be used for the illustrative analyte measurement devices and measurement methods.

### DETAILED DESCRIPTION

Various embodiments of a lactate concentration measurement device and corresponding operating methods enable real-time continuous hyperlactatemia monitoring that enable early detection of poor perfusion, typically attributable to shock, or for early detection of an acute inflammation disorder such as sepsis, Acute Lung Injury (ALI), the Acute Respiratory Distress Syndrome (ARDS), Multiple Organ Failure (MOF), or the like. These conditions can present dire health consequences to a patient, so the earlier detection is possible and precisely monitored, the better the opportunity for improving patient outcomes. A real-time lactate monitor can also greatly reduce the associated cost for lactate level measurement.

An additional benefit of a capability to monitor lactate as depicted herein can be assessment of the effect of various treatments that are given to alter the underlying conditions contributing to increases in lactate. For example, a decrease in lactate typically indicates that perfusion has been improved in a patient known to have been in shock. Alternatively, if the shock is not effectively corrected by administration of fluids or vasopressive agents, then lactate levels might not decrease, indicating a different therapeutic strategy, for example administration of more fluids, vasopressive agents, and/or an entirely new approach.

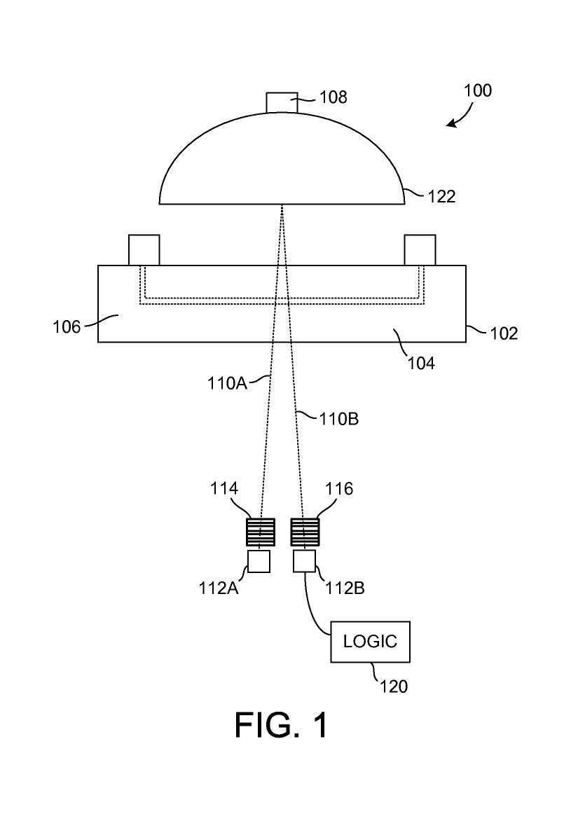

Referring to FIG. 1, a schematic block and pictorial diagram depicts an embodiment of a lactate concentration measurement apparatus 100 comprising a housing 102 enclosing a sample chamber 104 configured for holding a body fluid sample 106, an emitter 108 that emits light into the sample chamber 104, and a plurality of detectors 112 positioned along optical paths 110 across the sample chamber 104 from the emitter 108 that detect emitted light intensity. The measurement apparatus 100 further comprises measurement photo-optics 111 that generate light and monitor light intensity along multiple optical paths 110 in the sample chamber 104. Multiple optical filters 114,116 are aligned in respective paths of the multiple optical paths 110 including at least a first optical filter 114 with light absorption by lactate and water, and a second optical filter 116 with light absorption to water alone. A logic 120 determines the analyte concentration based on a ratio of intensities detected at a first detector 112A in an optical path 10A intersected by the first optical filter 114 and detected at a second detector 112B in an optical path 110B intersected by the second optical filter 116.

As shown, the measurement photo-optics 111 can comprise an emitter 108 that emits light into the sample chamber 104, and a plurality of detectors 112 positioned along respective optical paths 110 across the sample chamber 104 from the emitter 108 that detect emitted light intensity.

In an illustrative embodiment, the lactate concentration measurement apparatus 100 can have a first optical filter 114 comprising a filter λ1 with light absorption by lactate and water and a second optical filter 114 comprising a filter λ2 with light absorption by water alone. The logic 120 determines lactate concentration in the body fluid sample according to equation (1) as follows:

$${C}_{L} = \frac{\frac{{ɛ}_{{w}_{λ 1}}}{{ɛ}_{{w}_{λ 2}}} ln ( \frac{{I}_{{1}_{λ 2}}}{{I}_{{0}_{λ 2}}} ) - ln ( \frac{{I}_{{1}_{λ 1}}}{{I}_{{0}_{λ 1}}} )}{L {ɛ}_{{L}_{λ 1}}} , \qquad ( 1 )$$

where C_{L} is lactate molar fraction, L is path length through the body fluid sample, ε_{Lλ1} is lactate absorption coefficient at wavelength λ1, ε_{Wλ1} is water absorption coefficient at wavelength λ1, and ε_{Wλ2} is water absorption coefficient at wavelength λ2. I_{1λ1} is measured light intensity of wavelength λ1 through the body fluid sample, I_{0λ1} is light intensity of wavelength λ1 in absence of a sample in the sample chamber, I_{1λ2} is light intensity of wavelength λ2 through the body fluid sample in the sample chamber, and I_{0λ2} is light intensity of wavelength λ2 in absence of a sample 106 in the sample chamber 104.

In a first example application, the first optical filter 114 can be implemented as a filter with a light absorption wavelength λ1 of approximately 8.9 micrometers and the second optical filter 116 can be constructed as a filter with a light absorption wavelength λ2 of approximately 8.4 micrometers.

In a second example application, the first optical filter 114 can be implemented as a filter with a light absorption wavelength λ1 of approximately 8.3 micrometers and the second optical filter 116 can be constructed as a filter with a light absorption wavelength λ2 of approximately 8.4 micrometers.

In a particular arrangement, the first 114 and second 116 optical filters can be implemented as narrowband filters with a center wavelength variability of ±2%, a half power bandwidth of 0.12 micrometers, and peak transmission of 85%.

In various applications the housing 102 can enclose a sample chamber 104 configured for holding a body fluid sample such as plasma, serum, saliva, cerebrospinal fluid, tears, urine, extracellular fluids, or other fluid from a body that does not contain red blood cells or hemoglobin.

An implementation of a suitable housing 102 enclosing the sample chamber 104 can be formed of a material that is nonabsorbent to 8-10 micrometer light and is sufficiently rigid to maintain 10-50 micrometer spacing, and remains solid when contacted by body fluid. In a specific example, the housing 102 can be formed of high density polyethylene (HDPE) that has a transmission of approximately 53% at approximately 8.3, 8.4 and 8.9 micrometers.

In some embodiments, the measurement apparatus 100 can further comprise the emitter 108 in a configuration for radiating broadband infrared light, a parabolic reflector 122 which is separated by an air gap 124 from the emitter 108 and collimates the radiated broadband infrared light, and multiple detectors 112 positioned along respective optical paths 110 across the sample chamber 104 from the emitter 108 that detect emitted light intensity. The housing 102, emitter 108, and detectors 112 can be arranged so that the length of the optical paths 110 is in a range of approximately 10-50 micrometers.

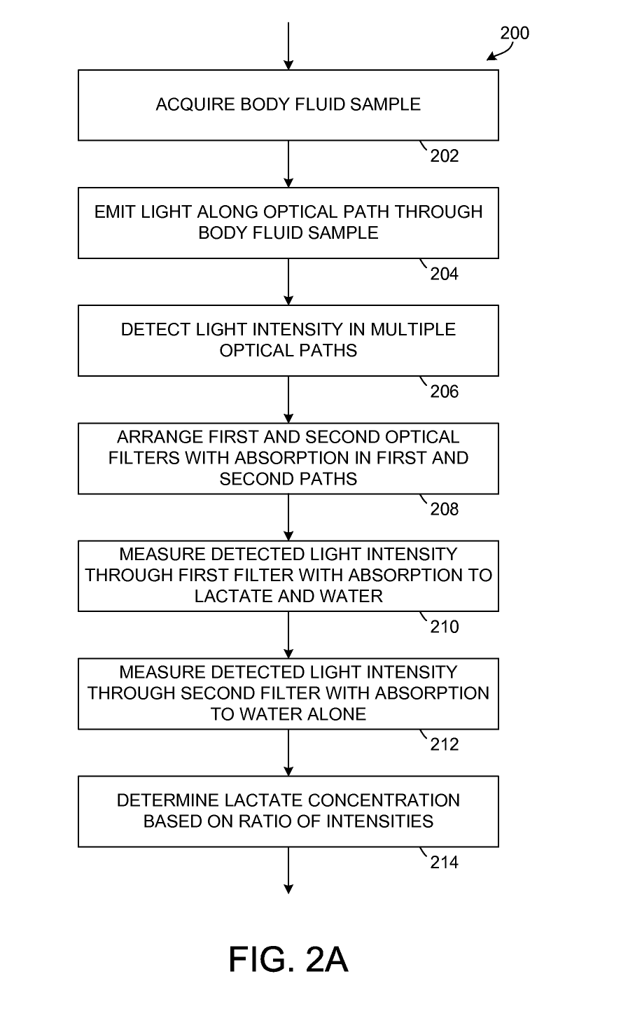

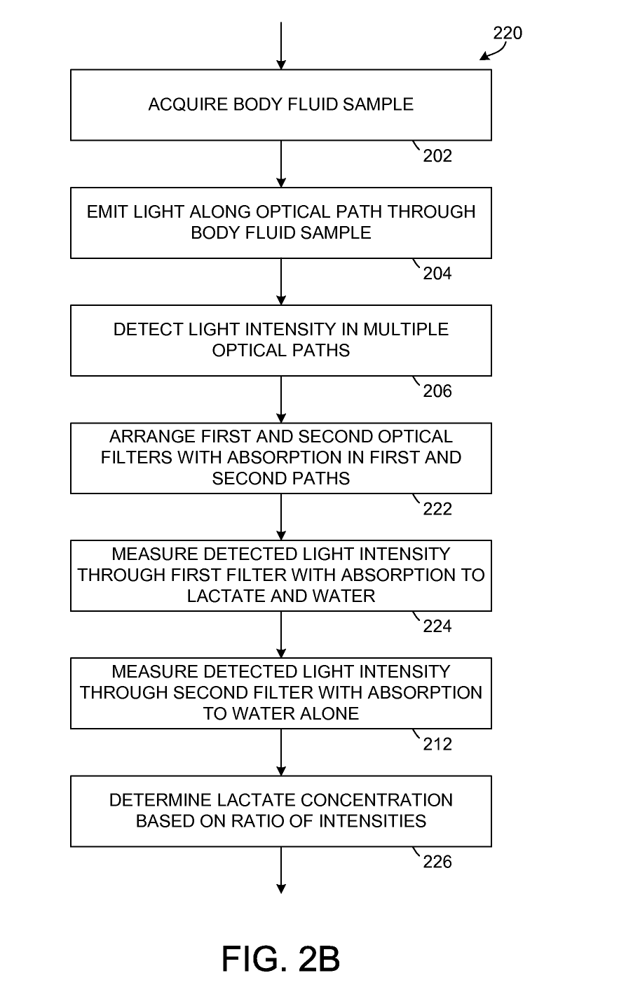

Referring to FIGS. 2A through 2B, flow charts illustrate one or more embodiments or aspects of a method for measuring 200 concentration of lactate in body fluid. As shown in FIG. 2A, the analyte concentration measurement method 200 comprises acquiring 202 a body fluid sample, emitting 204 light into the body fluid sample, and detecting 206 emitted light intensity on multiple optical paths through the body fluid sample. Multiple optical filters are arranged 208 in respective optical paths of the optical paths including at least a first optical filter with light absorption by lactate and water and a second optical filter with light absorption to water alone. The detected light intensity which is passed through the first optical filter is measured 210 and the detected light intensity which is passed through the second optical filter is measured 212. Analyte concentration is determined 214 based on a ratio of intensities detected at a detector in an optical path intersected by the first optical filter and detected at a detector in an optical path intersected by the second optical filter.

Referring to FIG. 2B, in a particular application lactate concentration can be measured 220 in body fluid by arranging 222 the optical filters including the first optical filter λ1 with light absorption by lactate and water, and the second optical filter λ2 with light absorption by water alone. Light intensity through the first filter with absorption to lactate and water is measured 224 and lactate concentration in the body fluid sample is determined 226 according to equation (1).

The concentration of lactate in a body fluid sample can be expressed by Beer's Law as shown in equation (2):

$${C}_{L} L {ɛ}_{λ} = - ln ( \frac{{I}_{1}}{{I}_{0}} ) , \qquad ( 2 )$$

where C_{L} is the lactate molar fraction, L is the path length, ε_{Lλ1} is the lactate absorption coefficient at wavelength λ with units of cm⁻¹, I₀ is the light intensity of wavelength λ at the detector for no sample and I₁ is the light intensity of wavelength at the detector for a sample. However, usage of equation (2) is not practical for diagnostic purposes because other analytes present in body fluid, such as water, albumin, lipids and urea, also absorb infrared light. A wavelength that avoids water absorption cannot be selected due to water's high concentration in body fluid and a strong absorbance throughout the infrared region. Equation (3) describes Beer's Law for wavelength λ1 where only lactate and water have absorption:

$${C}_{L} L {ɛ}_{{L}_{λ 1}} + {C}_{w} L {ɛ}_{{w}_{λ 1}} = - ln ( \frac{{I}_{{1}_{λ 1}}}{{I}_{{0}_{λ 1}}} ) , \qquad ( 3 )$$

where C_{L} is the lactate molar fraction, ε_{Lλ1} is the lactate absorption coefficient at wavelength λ1, C_{w} is the water molar fraction, ε_{wλ1} is the water absorption coefficient at wavelength λ1, I_{0λ1} is the light intensity of wavelength λ1 at the detector for no sample and I_{1λ1} is the light intensity of wavelength λ1 at the detector for a sample. The water concentration can be determined as shown in equation (4) by measuring the light absorption at wavelength λ2 where only water has absorption:

$${C}_{w} L {ɛ}_{{w}_{λ 2}} = - ln ( \frac{{I}_{{1}_{λ 2}}}{{I}_{{0}_{λ 2}}} ) , \qquad ( 4 )$$

Lactate concentration is determined by passing light through the sample and through filter λ1. Light is also passed through the sample and through filter λ2. The wavelength for filter λ1 is selected to have absorption by both lactate and water while filter λ2 has only water absorption. Lactate concentration is found by substituting equation (4) into equation (3) to result in equation (5) as follows:

$${C}_{L} = \frac{\frac{{ɛ}_{{w}_{λ 1}}}{{ɛ}_{{w}_{λ 2}}} ln ( \frac{{I}_{{1}_{λ 2}}}{{I}_{{0}_{λ 2}}} ) - ln ( \frac{{I}_{{1}_{λ 1}}}{{I}_{{0}_{λ 1}}} )}{L {ɛ}_{{L}_{λ 1}}} . \qquad ( 5 )$$

Filters can be selected to enhance measurement of lactate. For example, lactate has an absorption peak at 8.9 micrometers. The 8.4 micrometer wavelength is selected for the λ2 filter because there is no absorption exists at the wavelength except for water. Filters can be narrowband to avoid interference from nearby analytes. Center wavelengths of the filters can have a tolerance range of ±2%, the half power bandwidth 0.12 micrometers, and the peak transmission 85%.

Similarly the sample chamber material can also be selected to improve measurement of the selected analyte. For example, the sample chamber material can be selected which does not absorb 8-10 micrometer light, is sufficiently rigid to hold 10-50 micrometer spacing, and does not dissolve when contacted by body fluid. Zinc selenide meets all the criteria but is expensive and difficult to clean. A sample chamber material that is low cost and disposable may be more desirable. One suitable such material is high density polyethylene (HDPE) that has a transmission of 53% at 8.4 and 9.0 micrometers.

The sample also can be selected to facilitate measurement of the lactate. Plasma is a highly suitable sample due to abundance and an ability to be obtained at the patient bedside. Plasma is one of several body fluids that may be used as the sample. Other body fluids include serum, saliva, cerebrospinal fluids, tears, urine, extracellular fluids and any other fluids taken from the human body that do not contain red blood cells (RBCs) or hemoglobin. RBCs have a variable index of refraction and interfere with absorption measurements. Lactate level, oxygenation level, pH and temperature are some of the factors affecting RBC index of refraction. Hemoglobin absorbs light at 9.0 micrometers and interferes with lactate measurement at that wavelength.

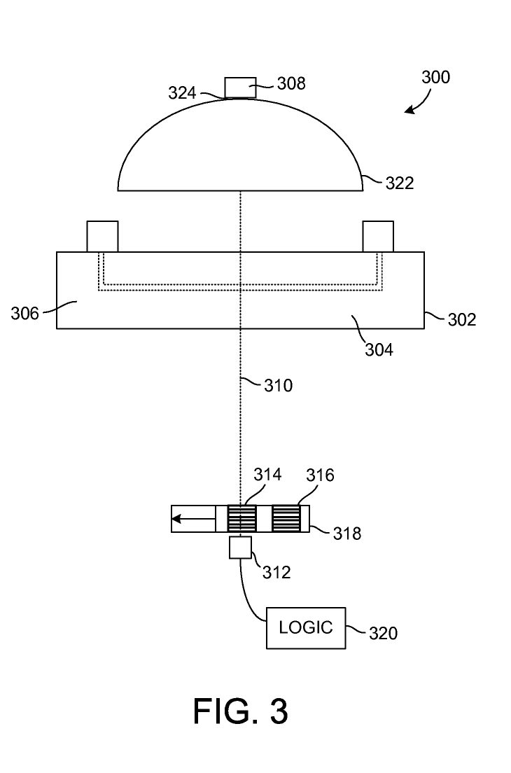

Referring to FIG. 3, a schematic block and pictorial diagram depicts an embodiment of a lactate concentration measurement apparatus 300 comprising a housing 302 enclosing a sample chamber 304 configured for holding a body fluid sample 306, measurement photo-optics 311 that generate light and monitor light intensity along an optical path 310 in the sample chamber. The measurement apparatus 300 further comprises a first optical filter 314 with light absorption by lactate and water, and a second optical filter 316 with light absorption to water alone. A switch 318 alternately interposes the first optical filter 314 and the second optical filter 316 into the optical path 310. A logic 320 determines the analyte concentration based on a ratio of intensities detected with the first optical filter 314 and the second optical filter 316 interposed into the optical path 310.

The measurement photo-optics 311 can comprise an emitter 308 that emits light along an optical path 310 into the sample chamber 304, and a detector 312 positioned along the optical path 310 across the sample chamber 304 from the emitter 308 that detects emitted light intensity.

In a particular application, the apparatus 300 can comprise a lactate concentration measurement apparatus with the first optical filter 314 comprising a filter λ1 with light absorption by lactate and water, and the second optical filter 316 comprising a filter λ2 with light absorption by water alone.

The switch 318 can be implemented as a sliding filter holder 326 whereby light passes through the selected filters 314, 316 held by the sliding filter holder 326 over the detector 312.

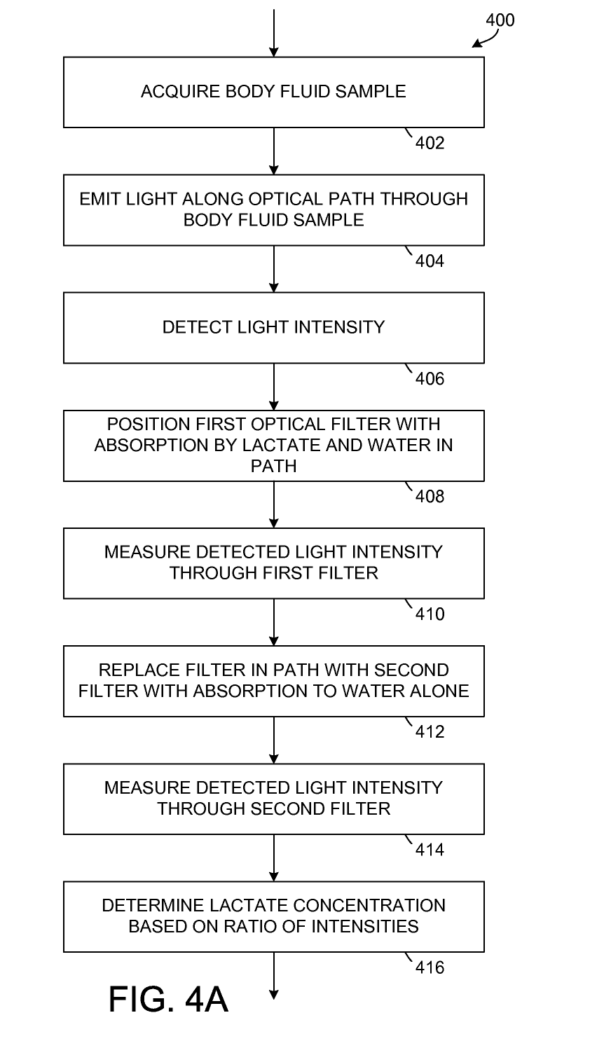

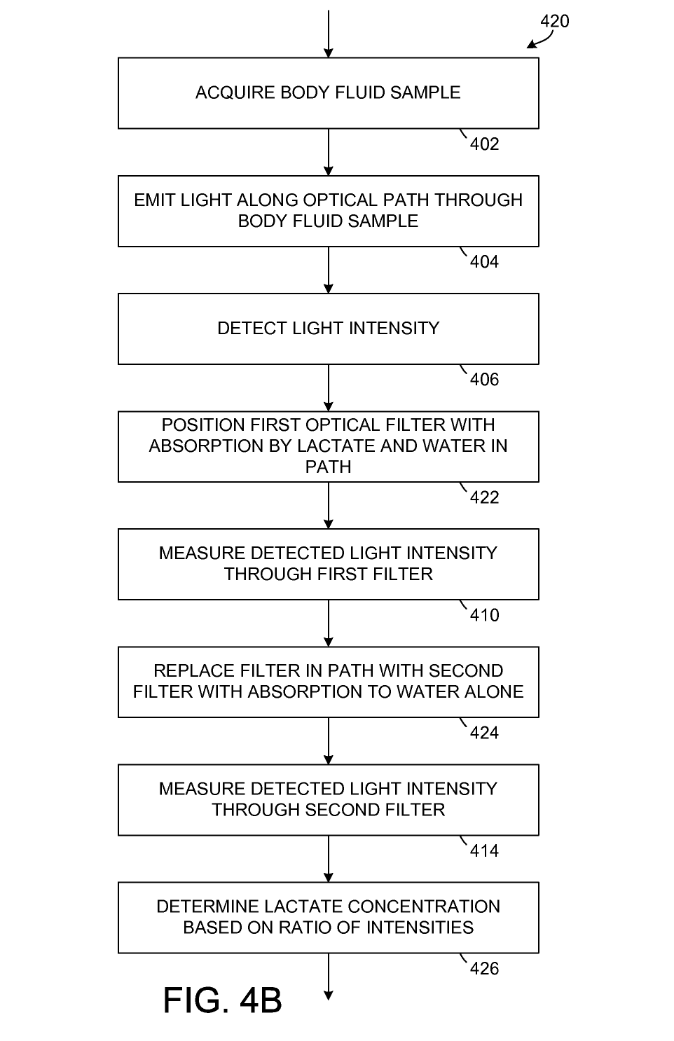

Referring to FIGS. 4A through 4B, flow charts illustrate one or more embodiments or aspects of a method for measuring 400 concentration of lactate in body fluid. As shown in FIG. 4A, the analyte concentration measurement method 400 comprises acquiring 402 a body fluid sample, emitting 404 light along an optical path into the body fluid sample, and detecting 406 emitted light intensity on the optical path through the body fluid sample. A first optical filter with light absorption by lactate and water can be positioned 408 in the optical path and the detected light intensity passed through the first optical filter is measured 410. The first optical filter can be replaced 412 with a second optical filter with light absorption to water alone and the detected light intensity passed through the second optical filter is measured 414. Analyte concentration is determined 416 based on a ratio of intensities detected with the first optical filter and the second optical filter interposed into the optical path.

The acquired body fluid sample can be, for example, plasma, serum, saliva, cerebrospinal fluid, tears, urine, extracellular fluids, or other fluid from a body that does not contain red blood cells or hemoglobin.

Referring to FIG. 4B, in a particular application lactate concentration can be measured 420 in body fluid by positioning 422 the first optical filter comprising a filter λ1 with light absorption by lactate and water, and replacing 424 the first optical filter with the second optical filter comprising a filter λ2 with light absorption by water alone. Lactate concentration in the body fluid sample is determined 426 according to equation (1).

In various applications, the first optical filter can be implemented as a filter with a light absorption wavelength λ1 of approximately 8.9 or 8.3 micrometers, for example, and the second optical filter with a light absorption wavelength λ2 of approximately 8.4 micrometers.

Referring again to FIG. 3, a schematic block diagram depicts an embodiment of an illustrative infrared lactate sensor 300. The infrared lactate sensor 300 measures lactate concentration in a body fluid sample 306 by determining the portion of light absorbed by lactate. An emitter 308 radiates broadband infrared light that is collimated with a parabolic shaped reflector 322. In an illustrative implementation, a 10 millimeter (mm) air gap is interposed between the reflector 322 and a sample chamber 304 to minimize thermal influences. The path length through the sample can be configured in a range between 10-50 micrometers (μm) to reduce or eliminate effects of strong water absorption. Light passes through one of two filters 314, 316 held by a sliding filter holder 318 over a detector 312. An alternate embodiment shown in FIG. 3 has two detectors, each with a filter.

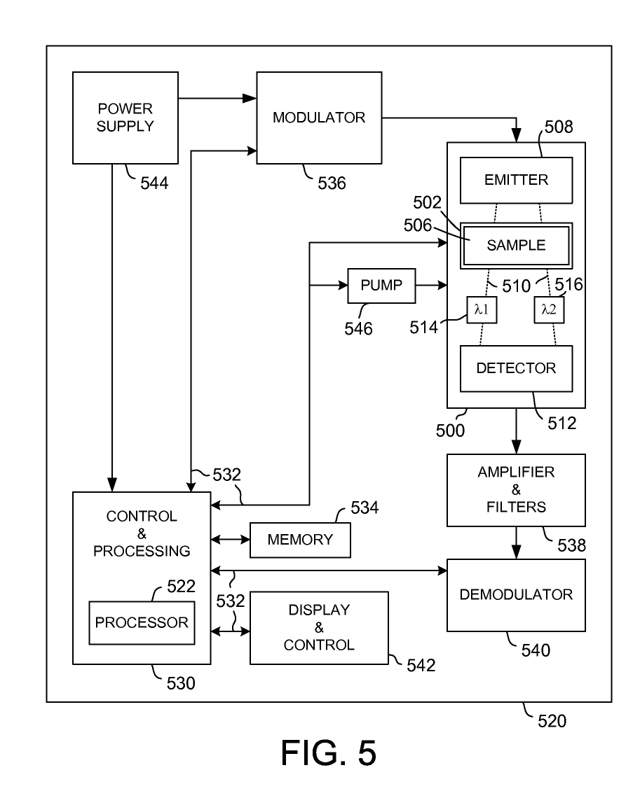

Referring to FIG. 5, a schematic block diagram illustrates an embodiment of a system 520 that can be used for the illustrative analyte measurement devices and measurement methods.

An embodiment of a lactate concentration measurement apparatus 500 can further comprise a display 542 coupled to the housing 502 so that a lactate measurement is locally determined and displayed within the housing 502 and the body fluid sample 506 is continuously contained within a closed loop including the lactate concentration measurement apparatus 500 and a patient's body.

Also some embodiments of the lactate concentration measurement apparatus 500 can support real-time monitoring and analysis. The apparatus 500 can comprise a display 542 coupled to the housing 502 and a logic 522 that determines a lactate measurement in real-time for real-time presentation on the display 542.

Furthermore, some implementations of the lactate concentration measurement apparatus 500 can function in an automatic control system. For example, a system 520 can comprise a fluid loop that couples the sample chamber 504 to a patient's body fluid system and a controllable infusion pump 546 coupled to the fluid loop. The logic 522 controls, with logic automation, the infusion pump 546 for administration of therapeutic fluids into the fluid loop based on the lactate concentration.

In an illustrative embodiment, a control and processing board 530 supports control/processing operations. A processor 522 controls the sensor 500 and supports hardware through an I²C serial bus 532. The processor 522 measures the light intensity I_{1λ1} for 10 seconds with filter λ1 514 in the optical path 510, moves filter λ2 516 into the optical path 510, measures the light intensity I_{1λ2} for 10 seconds with filter λ2 516, computes the average of both intensities and calculates the lactate concentration using equation (5). Lactate concentration is displayed and stored in combination with the 10 second intensities on a secure digital (SD) memory card 534.

An illustrative system 520 also includes a modulator 536. The modulator 536 uses a 2.0 megahertz (MHz) crystal oscillator-produced square wave signal and passes the signal through a series of counters to divide down to a 7-8 hertz (Hz) square wave. The reduced-frequency square wave is used to turn the emitter 508 on and off, allowing a periodic change in the light intensity on the detector 512.

The emitter 508 can be implemented as a broadband mid-infrared source that emits light over the 1-20 micrometer range. Intex MIRL 17-900-R is an emitter device that meets the criteria and has a parabolic reflector built into the device directly behind the emitter to collimate and focus the emitted light. The drive voltage for the emitter 508 can be supplied by a LT1129 programmable linear regulator.

A detector 512 converts changes in incident infrared energy into voltage. A suitable detector 512 is the InfraTech LIE-345 pyroelectric detector.

A signal from the detector 512 can be passed to an amplifier and filters 538 including a notch filter at 60 Hz to reduce 60 Hz noise induced by surrounding electrical sources. The notch filter has built in amplification under 60 Hz and a reduced gain above the 60 Hz notch. Amplification of the pre-notch and post-notch frequencies can be changed by changing resistor values. The output of the amplifier/filter 538 is fed into a demodulator 540.

The demodulator 540 receives the amplified and filtered detector output signal and converts the signal into a DC level by taking the difference between the on and off states of the square wave. The demodulator 540 suppresses voltage fluctuations outside the 7-8 Hz range. The demodulator 540 can have a programmable phase adjustment that allows the phase of the demodulator 540 to match the phase of the modulator 536. The output signal from the demodulator 540 is sampled using an analog-to-digital converter.

The illustrative system 520 further comprises a power supply 544 which can be a switching 85-264V RMS 47-63 Hz AC to 12V DC 7 watt medical grade power supply.

Terms “substantially”, “essentially”, or “approximately”, that may be used herein, relate to an industry-accepted tolerance to the corresponding term. Such an industry-accepted tolerance ranges from less than one percent to twenty percent and corresponds to, but is not limited to, functionality, values, process variations, sizes, operating speeds, and the like. The term “coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. Inferred coupling, for example where one element is coupled to another element by inference, includes direct and indirect coupling between two elements in the same manner as “coupled”.

The illustrative block diagrams and flow charts depict process steps or blocks that may represent modules, segments, or portions of code that include one or more executable instructions for implementing specific logical functions or steps in the process. Although the particular examples illustrate specific process steps or acts, many alternative implementations are possible and commonly made by simple design choice. Acts and steps may be executed in different order from the specific description herein, based on considerations of function, purpose, conformance to standard, legacy structure, and the like.

While the present disclosure describes various embodiments, these embodiments are to be understood as illustrative and do not limit the claim scope. Many variations, modifications, additions and improvements of the described embodiments are possible. For example, those having ordinary skill in the art will readily implement the steps necessary to provide the structures and methods disclosed herein, and will understand that the process parameters, materials, and dimensions are given by way of example only. The parameters, materials, and dimensions can be varied to achieve the desired structure as well as modifications, which are within the scope of the claims. Variations and modifications of the embodiments disclosed herein may also be made while remaining within the scope of the following claims.

What is claimed is:

1. A lactate concentration measurement apparatus comprising:
a housing enclosing a sample chamber configured for holding a body fluid sample;
measurement photo-optics that generate light and monitor light intensity along a plurality of optical paths in the sample chamber;
a plurality of optical filters aligned in respective optical paths of the optical path plurality comprising at least a first optical filter with light absorption by lactate and water and a second optical filter with light absorption to water alone; and
a logic that determines the lactate concentration based on a ratio of intensities detected at a first detector in an optical path intersected by the first optical filter and detected at a second detector in an optical path intersected by the second optical filter.

2. The apparatus according to claim 1 further comprising:
the measurement photo-optics comprise:
an emitter that emits light into the sample chamber; and
a plurality of detectors positioned along respective optical paths across the sample chamber from the emitter that detect emitted light intensity.

3. The apparatus according to claim 1 further comprising:
the first optical filter comprising a filter λ1 with light absorption by lactate and water;
the second optical filter comprising a filter λ2 with light absorption by water alone; and
the logic determines lactate concentration in the body fluid sample according to an equation as follows:
$${C}_{L} = \frac{\frac{{ɛ}_{{w}_{λ 1}}}{{ɛ}_{{w}_{λ 2}}} ln ( \frac{{I}_{{1}_{λ 2}}}{{I}_{{0}_{λ 2}}} ) - ln ( \frac{{I}_{{1}_{λ 1}}}{{I}_{{0}_{λ 1}}} )}{L {ɛ}_{{L}_{λ 1}}} ,$$
where C_{L} is lactate molar fraction, L is path length through the body fluid sample, ε_{Lλ1} is lactate absorption coefficient at wavelength λ1, ε_{Wλ1} is water absorption coefficient at wavelength λ1, ε_{Wλ2} is water absorption coefficient at wavelength λ2, ε_{1λ1} is measured light intensity of wavelength λ1 through the body fluid sample, I_{0λ1} is light intensity of wavelength λ1 in absence of a sample in the sample chamber, I_{1λ2} is light intensity of wavelength λ2 through the body fluid sample in the sample chamber, and I_{0λ2} is light intensity of wavelength λ2 in absence of a sample in the sample chamber.

4. The apparatus according to claim 3 further comprising:
the first optical filter comprising a filter with a light absorption wavelength λ1 of approximately 8.9 micrometers; and
the second optical filter comprising a filter with a light absorption wavelength λ2 of approximately 8.4 micrometers.

5. The apparatus according to claim 3 further comprising:
the first optical filter comprising a filter with a light absorption wavelength λ1 of approximately 8.3 micrometers; and
the second optical filter comprising a filter with a light absorption wavelength λ2 of approximately 8.4 micrometers.

6. The apparatus according to claim 1 further comprising:
the housing enclosing a sample chamber configured for holding a body fluid sample comprising plasma, serum, saliva, cerebrospinal fluid, tears, urine, extracellular fluids, or other fluid from a body that does not contain red blood cells or hemoglobin.

7. The apparatus according to claim 1 further comprising:
the measurement photo-optics comprising:
the emitter configured to radiate broadband infrared light;
a parabolic reflector separated by an air gap from the emitter that collimates the radiated broadband infrared light; and
a plurality of detectors positioned along respective optical paths across the sample chamber from the emitter that detect emitted light intensity, wherein
the housing emitter, and detector plurality are arranged whereby optical path lengths are in an approximate range of 10-50 micrometers.

8. The apparatus according to claim 1 further comprising:
the first and second optical filters comprising narrowband filters with a center wavelength variability of ±2%, a half power bandwidth of 0.12 micrometers, and peak transmission of 85%.

9. The apparatus according to claim 1 further comprising:
the housing enclosing the sample chamber is formed of a material that is nonabsorbent to 8-10 micrometer light and is sufficiently rigid to maintain 10-50 micrometer spacing, and remains solid when contacted by body fluid.

10. The apparatus according to claim 1 further comprising:
the housing enclosing the sample chamber is formed of high density polyethylene (HDPE) that has a transmission of approximately 53% at approximately 8.3, 8.4 and 8.9 micrometers.

11. The apparatus according to claim 1 further comprising:
a display coupled to the housing whereby a lactate measurement is locally determined and displayed within the housing and the body fluid sample is continuously contained within a closed loop including the lactate concentration measurement apparatus and a patient's body.

12. The apparatus according to claim 1 further comprising:
a display coupled to the housing; and
the logic that determines a lactate measurement in real-time for real-time presentation on the display.

13. The apparatus according to claim 1 further comprising:
a fluid loop that couples the sample chamber to a patient's body fluid system;
a controllable infusion pump coupled to the fluid loop; and
the logic that controls, with logic automation, the infusion pump for administration of therapeutic fluids into the fluid loop based on the lactate concentration.

14. A method for measuring concentration of lactate in body fluid comprising:
acquiring a body fluid sample;
emitting light into the body fluid sample;
detecting emitted light intensity on a plurality of optical paths through the body fluid sample;
arranging a plurality of optical filters in respective optical paths of the optical path plurality comprising at least a first optical filter with light absorption by lactate and water and a second optical filter with light absorption to water alone;
measuring the detected light intensity passed through the first optical filter;
measuring the detected light intensity passed through the second optical filter;
determining lactate concentration based on a ratio of intensities detected at a detector in an optical path intersected by the first optical filter and detected at a detector in an optical path intersected by the second optical filter.

15. The method according to claim 14 further comprising:
measuring lactate concentration in body fluid;
arranging the optical filters including the first optical filter comprising a filter λ1 with light absorption by lactate and water, and the second optical filter comprising a filter λ2 with light absorption by water alone; and
determining lactate concentration in the body fluid sample according to an equation as follows:
$${C}_{L} = \frac{\frac{{ɛ}_{{w}_{λ 1}}}{{ɛ}_{{w}_{λ 2}}} ln ( \frac{{I}_{{1}_{λ 2}}}{{I}_{{0}_{λ 2}}} ) - ln ( \frac{{I}_{{1}_{λ 1}}}{{I}_{{0}_{λ 1}}} )}{L {ɛ}_{{L}_{λ 1}}} ,$$
where C_{L} is lactate molar fraction, L is path length through the body fluid sample, ε_{Lλ1} is lactate absorption coefficient at wavelength λ1, ε_{Wλ1} is water absorption coefficient at wavelength λ1, ε_{Wλ2} is water absorption coefficient at wavelength λ2, I_{1λ1} is measured light intensity of wavelength λ1 through the body fluid sample, I_{0λ1} is light intensity of wavelength λ1 in absence of a sample in the sample chamber, I_{1λ2} is light intensity of wavelength λ2 through the body fluid sample in the sample chamber, and I_{0λ2} is light intensity of wavelength λ2 in absence of a sample in the sample chamber.

16. The method according to claim 15 further wherein:
the first optical filter comprises a filter with a light absorption wavelength λ1 of approximately 8.9 micrometers; and
the second optical filter comprises a filter with a light absorption wavelength λ2 of approximately 8.4 micrometers.

17. The method according to claim 15 wherein:
the first optical filter comprises a filter with a light absorption wavelength λ1 of approximately 8.3 micrometers; and
the second optical filter comprises a filter with a light absorption wavelength λ2 of approximately 8.4 micrometers.

18. The method according to claim 14 further comprising:
acquiring the body fluid sample comprising plasma, serum, saliva, cerebrospinal fluid, tears, urine, extracellular fluids, or other fluid from a body that does not contain red blood cells or hemoglobin.

19. The method according to claim 14 further comprising:
acquiring the body fluid sample in a closed body loop;
locally determining a lactate measurement in the closed body loop; and
displaying the lactate measurement local to the closed body loop.

20. The method according to claim 14 further comprising:
determining a lactate measurement in real-time; and
displaying the lactate measurement in real-time.

21. The method according to claim 14 further comprising:
acquiring the body fluid sample in a closed body loop;
pumping a therapeutic fluid into the closed body loop; and
controlling, with logic automation, pumping for administration of therapeutic fluids into the fluid loop based on the lactate concentration.

22. A lactate concentration measurement apparatus comprising:
a housing enclosing a sample chamber configured for holding a body fluid sample;
measurement photo-optics that generate light and monitor light intensity along an optical path in the sample chamber;
a first optical filter with light absorption by a lactate and water;
a second optical filter with light absorption to water alone;
a switch that alternately interposes the first optical filter and the second optical filter into the optical path; and
a logic that determines lactate concentration based on a ratio of intensities detected with the first optical filter and the second optical filter interposed into the optical path.

23. The apparatus according to claim 22 further comprising:
the measurement photo-optics comprising:
an emitter that emits light along an optical path into the sample chamber; and
a detector positioned along the optical path across the sample chamber from the emitter that detects emitted light intensity.

24. The apparatus according to claim 22 further comprising:
the first optical filter comprising a filter λ1 with light absorption by lactate and water;
the second optical filter comprising a filter λ2 with light absorption by water alone; and
the logic determines lactate concentration in the body fluid sample according to an equation as follows:
$${C}_{L} = \frac{\frac{{ɛ}_{{w}_{λ 1}}}{{ɛ}_{{w}_{λ 2}}} ln ( \frac{{I}_{{1}_{λ 2}}}{{I}_{{0}_{λ 2}}} ) - ln ( \frac{{I}_{{1}_{λ 1}}}{{I}_{{0}_{λ 1}}} )}{L {ɛ}_{{L}_{λ 1}}} ,$$
where C_{L} is lactate molar fraction, L is path length through the body fluid sample, ε_{Lλ1} is lactate absorption coefficient at wavelength λ1, ε_{Wλ1} is water absorption coefficient at wavelength λ1, ε_{Wλ2} is water absorption coefficient at wavelength λ2, I_{1λ1} is measured light intensity of wavelength λ1 through the body fluid sample, I_{0λ1} is light intensity of wavelength λ1 in absence of a sample in the sample chamber, I_{1λ2} is light intensity of wavelength λ2 through the body fluid sample in the sample chamber, and I_{0λ2} is light intensity of wavelength λ2 in absence of a sample in the sample chamber.

25. The apparatus according to claim 24 further comprising:
the first optical filter comprising a filter with a light absorption wavelength λ1 of approximately 8.9 micrometers; and
the second optical filter comprising a filter with a light absorption wavelength λ2 of approximately 8.4 micrometers.

26. The apparatus according to claim 24 further comprising:
the first optical filter comprising a filter with a light absorption wavelength λ1 of approximately 8.3 micrometers; and
the second optical filter comprising a filter with a light absorption wavelength λ2 of approximately 8.4 micrometers.

27. The apparatus according to claim 22 further comprising:
the housing enclosing a sample chamber configured for holding a body fluid sample comprising plasma, serum, saliva, cerebrospinal fluid, tears, urine, extracellular fluids, or other fluid from a body that does not contain red blood cells or hemoglobin.

28. The apparatus according to claim 22 further comprising:
the measurement photo-optics comprising:
an emitter configured to radiate broadband infrared light;
a parabolic reflector separated by an air gap from the emitter that collimates the radiated broadband infrared light; and
a detector; wherein
the housing, emitter, and detector are arranged whereby optical path length is in an approximate range of 10-50 micrometers.

29. The apparatus according to claim 22 further comprising:
the switch comprising a sliding filter holder whereby light passes through a selected filters held by the sliding filter holder over the detector.

30. The apparatus according to claim 22 further comprising:
the first and second optical filters comprising narrowband filters with a center wavelength variability of ±2%, a half power bandwidth of 0.12 micrometers, and peak transmission of 85%.

31. The apparatus according to claim 22 further comprising:
the housing enclosing the sample chamber is formed of a material that is nonabsorbent to 8-10 micrometer light and is sufficiently rigid to maintain 10-50 micrometer spacing, and remains solid when contacted by body fluid.

32. The apparatus according to claim 22 further comprising:
the housing enclosing the sample chamber is formed of high density polyethylene (HDPE) that has a transmission of approximately 53% at approximately 8.3, 8.4 and 8.9 micrometers.

33. The apparatus according to claim 22 further comprising:
a display coupled to the housing whereby a lactate measurement is locally determined and displayed within the housing and the body fluid sample is continuously contained within a closed loop including the lactate concentration measurement apparatus and a patient's body.

34. The apparatus according to claim 22 further comprising:
a display coupled to the housing; and
the logic that determines a lactate measurement in real-time for real-time presentation on the display.

35. The apparatus according to claim 22 further comprising:
a fluid loop that couples the sample chamber to a patient's body fluid system;
a controllable infusion pump coupled to the fluid loop; and
the logic that controls, with logic automation, the infusion pump for administration of therapeutic fluids into the fluid loop based on the lactate concentration.

36. A method for measuring concentration of lactate concentration in body fluid comprising:
acquiring a body fluid sample;
emitting light along an optical path into the body fluid sample;
detecting emitted light intensity on the optical path through the body fluid sample;
positioning a first optical filter with light absorption by lactate and water in the optical path;
measuring the detected light intensity passed through the first optical filter;
replacing the first optical filter with a second optical filter with light absorption to water alone;
measuring the detected light intensity passed through the second optical filter; and
determining lactate concentration based on a ratio of intensities detected with the first optical filter and the second optical filter interposed into the optical path.

37. The method according to claim 36 further comprising:
measuring lactate concentration in body fluid;
positioning the first optical filter comprising a filter λ1 with light absorption by lactate and water;
replacing the first optical filter with the second optical filter comprising a filter λ2 with light absorption by water alone; and
determining lactate concentration in the body fluid sample according to an equation as follows:
$${C}_{L} = \frac{\frac{{ɛ}_{{w}_{λ 1}}}{{ɛ}_{{w}_{λ 2}}} ln ( \frac{{I}_{{1}_{λ 2}}}{{I}_{{0}_{λ 2}}} ) - ln ( \frac{{I}_{{1}_{λ 1}}}{{I}_{{0}_{λ 1}}} )}{L {ɛ}_{{L}_{λ 1}}} ,$$
where C_{L} is lactate molar fraction, L is path length through the body fluid sample, ε_{Lλ1} is lactate absorption coefficient at wavelength λ1, ε_{Wλ1} is water absorption coefficient at wavelength λ1, ε_{Wλ2} is water absorption coefficient at wavelength λ2, I_{1λ1} is measured light intensity of wavelength λ1 through the body fluid sample, I_{0λ1} is light intensity of wavelength λ1 in absence of a sample in the sample chamber, I_{1λ2} is light intensity of wavelength λ2 through the body fluid sample in the sample chamber, and I_{0λ2} is light intensity of wavelength λ2 in absence of a sample in the sample chamber.

38. The method according to claim 37 further wherein:
the first optical filter comprises a filter with a light absorption wavelength λ1 of approximately 8.9 micrometers; and
the second optical filter comprises a filter with a light absorption wavelength λ2 of approximately 8.4 micrometers.

39. The method according to claim 37 wherein:
the first optical filter comprises a filter with a light absorption wavelength λ1 of approximately 8.3 micrometers; and
the second optical filter comprises a filter with a light absorption wavelength λ2 of approximately 8.4 micrometers.

40. The method according to claim 36 further comprising:
acquiring the body fluid sample comprising plasma, serum, saliva, cerebrospinal fluid, tears, urine, extracellular fluids, or other fluid from a body that does not contain red blood cells or hemoglobin.

41. The method according to claim 36 further comprising:
acquiring the body fluid sample in a closed body loop;
locally determining a lactate measurement in the closed body loop; and
displaying the lactate measurement local to the closed body loop.

42. The method according to claim 36 further comprising:
determining a lactate measurement in real-time; and
displaying the lactate measurement in real-time.

43. The method according to claim 36 further comprising:
acquiring the body fluid sample in a closed body loop;
pumping a therapeutic fluid into the closed body loop; and
controlling, with logic automation, pumping for administration of therapeutic fluids into the fluid loop based on the lactate concentration.
